(19) United States
(12) Patent Application Publication
(10) Pub. No.: US 2006/0067954 A1
(43) Pub. Date: Mar. 30, 2006

(54) Lipid stabilized formulations

(76) Inventors: Ian W. Cottrell, Basking Ridge, NJ (US); Albert Ahn, Short Hills, NJ (US); Richard Fisher, Brookville, OH (US)

Correspondence Address: STROOCK & STROOCK & LAVAN LLP, 180 MAIDEN LANE, NEW YORK, NY, 10038, US

(21) Appl. No.: 11/228,120

(22) Filed: Sep. 16, 2005

Related U.S. Application Data
(60) Provisional application No. 60/612,761, filed on Sep. 24, 2004.

(51) Int. Cl. A61K 31/517; A61K 9/00; A61K 31/7048

(52) U.S. Cl. 424/400; 514/28; 514/266.22

(57) ABSTRACT

The present invention provides a lipid based system for isolating components in a pharmaceutical formulation. Also provided are methods for preparing stable pharmaceutical formulations containing at least two active ingredients. Further provided are methods of treating helminthiasis in mammals, which comprises administering a pharmaceutical composition that is highly effective against helminths, particularly tapeworm, hookworm, roundworm and heartworm of domestic animals and farm animals.

This application claims the benefit of U.S. Provisional Application No. 60/612,761, filed Sep. 24, 2004.

### BACKGROUND OF INVENTION

The invention relates generally to anthelmintic formulations which can have significant parasiticidal activity as anthelmintics in animal health and more particularly to solid anthelmintic formulations containing ivermectin.

Active ingredients of anthelmintics and their methods of formation in accordance with preferred embodiments of the invention are discussed in U.S. Pat. Nos. 3,502,661, 4,001,411 and 4,199,569, pending U.S. Ser. Nos. 10/637,807 and 10/800,407, and PCT/US04/025,005 entitled “Improved Anthelmintic Formulations, filed Aug. 3, 2004, the contents of which are incorporated herein by reference.

It is often beneficial, under certain circumstances, to include multiple drugs in the same formulation in order to target a wider variety of parasites. For example, a multidrug formulation may be useful in overcoming problems seen with single drug resistance. The inclusion of greater than one anthelmintic in the formulations discussed herein may have an increased likelihood of eliminating a particular helminth that is resistant to other included anthelmintic compounds. Accordingly, even if the helminth is resistant to one or two of the active ingredients, it is likely that at least one of the other active ingredients will be effective at eliminating the helminth in question.

The disease or group of diseases described generally as helminthiasis is due to infestation of an animal host with parasitic worms known as helminths. Helminthiasis is a prevalent and serious economic problem in domesticated animals such as swine, sheep, horses, cattle, goats, dogs, cats and poultry. Among the helminths, the group of worms described as nematodes causes widespread and often times serious infection in various species of animals. Still other parasites may be located in other tissues and organs of the body such as the heart and blood vessels, subcutaneous and lymphatic tissue and the like. The parasitic infections known as helminthiases lead to anemia, malnutrition, weakness, weight loss, severe damage to the walls of the intestinal tract and other tissues and organs and, if left untreated, may result in death of the infected host.

Examples of particularly desirable parasitical agents include ivermectin and praziquantel. However, ivermectin is hygroscopic and therefore tends to be undesirably unstable. It has also been determined that ivermectin is unstable in both acidic and basic solutions and is susceptible to photodegradation and oxidative degradation. Accordingly, it is very difficult to prepare a solid composition, such as a powder, tablet or pellet, containing ivermectin without having to resort to using a large amount of filler material to make up the bulk of the solid in order to maintain the integrity of the compound. Even then, degradation problems can exist.

This problem is compounded when additional actives are intended to be included in the same formulation, as the interaction of ivermectin with other actives can result in an unstable formulation. Thus, while it is desirable to formulate a composition containing ivermectin and other parasitical agents such as praziquantel, it can be difficult to formulate a stable multidrug composition that can be stored at room temperature for reasonable amounts of time.

U.S. Pat. No. 6,340,672 discloses a liquid solvent system for anthelmintics. However, it does not provide a solid formulation or address the problem of instability caused by interaction among the active ingredients. U.S. Pat. No. 4,597,969 discloses mixing an active compound such as ivermectin with a second ingredient and spray granulating the resulting formulation with alginic acid. However, alginic acid, being negatively charged, can bind to compounds having a positive charge such as pyrantel, which could impact the bioavailability of the anthelmintics.

Pending U.S. Ser. No. 10/637,807 discloses a solid anthelmintic formulation that achieves improvements stability through spray granulation with a neutral carrier instead of alginic acid. While this method improves stability, spray granulation methods disclosed therein can be difficult to scale-up for commercial production.

Accordingly, it is desirable to provide improved formulations and methods of preparing stable multidrug anthelmintic formulations which can be formed into a solid or tablet of optimal size, palatable to animals and can be easily administered to the affected animal.

### SUMMARY OF THE INVENTION

Generally speaking, in accordance with the invention, a pharmaceutical formulation and methods of preparation are provided for use in the treatment of helminthiasis of mammals, and particularly for the treatment of tapeworm, hookworm, roundworm and heartworm of domestic animals and farm animals.

Accordingly, the present invention provides a method of treating helminthiasis in mammals, which method comprises administering to the mammal in need thereof, an anthelmintically effective amount of a pharmaceutical formulation of the invention. The present invention also provides a method for preparing a suitably stable pharmaceutical formulation containing ivermectin in combination with other active compositions such as hexahydropyrazinoisoquinolines and anthelmintic pyrimidines such as tetrahydropyrimidines. Examples of these include, for example, praziquantel and pyrantel pamoate, respectively. Formulations in accordance with the invention can remain stable when stored at room temperature for over one month, and typically, much longer.

An aspect of the preferred method involves coating dry active parasitical ingredients with a lipid and forming a homogeneous liquid lipid suspension by maintaining the suspension at a sufficiently high temperature. The suspension containing the active ingredient is formed into the desired form by molding or pouring the suspension into the bottom of a suitable container or mold. The suspension can then be cooled to a solid condition.

In one preferred embodiment of the invention, a method of preparing a multidrug composition comprises the following steps:
(a) preparing a first and a second and/or optionally also a third liquid-like lipid suspensions comprising a first, second, and/or optionally a third active ingredient, respectively;
(b) pouring the first liquid-like lipid suspension into a container;
(c) permitting the first liquid-like lipid suspension to cool, thereby resulting in a first solid-like layer comprising the first active ingredient;
(d) optionally pouring the second liquid-like lipid suspension on the first solid-like layer;
(e) permitting the second liquid-like lipid suspension to cool, thereby resulting in a second solid-like layer located on top of the first solid-like layer;
(f) optionally pouring the third liquid-like lipid suspension on top of the second solid-like layer; and
(e) permitting the third liquid-like lipid suspension to cool, thereby resulting in a third solid-like layer located on top of the second solid-like layer, wherein the first active ingredient in the first solid-like layer is separated from the third active ingredient in the third solid-like layer by the second solid-like layer.

It is of course understood that when the method is used to produce a multidrug composition comprising two active ingredients that interact with other, the second liquid lipid suspension can be prepared without an active ingredient and placed between the first and third layers containing the active ingredients in order to prevent interaction between the active ingredients. In addition, formulations with four, five or even six layers could be acceptable. For example, a non-active lipid layer could be disposed between both the first and second layers, as well as between the second and third active layers.

Accordingly, it is an object of the invention to provide a single dosage multidrug composition having increased stability.

Another object of the invention is to provide a multidrug formulation that is effective against a variety of parasites.

Another object of the invention is to provide a method for producing a multidrug composition with increased stability.

Other objects and features will be in part apparent and in part pointed out.

### DETAILED DESCRIPTION OF THE PREFERRED EMBODIMENTS

The invention provides a lipid based system for isolating components in a pharmaceutical formulation. A liquid suspension is cooled to yield a solid. A lipid barrier layer is then disposed over the first layer to isolate the first layer. In accordance with the invention, a multidrug composition comprising at least two active ingredients, and preferably, three or more active ingredients can be provided. Preferably, the composition comprises a combination of avermectins, hexahydropyrazinoisoquinolines and anthelmintic pyrimidines such as tetrahydropyrimidines. Preferred tetrahydropyrimidines include, for example, pyrantel pamoate, morantel and oxantel. Preferred hexahydropyrazinoisoquinolines include, for example, praziquantel. Other acceptable actives include benzazepines and salicylamides. Preferred avermectins include, for example, ivermectin, doramectin, selamectin and abamectin.

The combination of avermectins, hexahydropyrazinoisoquinolines and anthelmintic pyrimidines target a wide variety of pathogenic organisms that can adversely affect the health of a mammal. However, administering three physically separate pharmaceutical compositions to an animal is undesirable and it has been determined that it would be beneficial to combine the ingredients into one formulation, in particular one tablet, capsule or packet containing a pharmaceutically effective amount of the active ingredients, thereby decreasing the number of administrations of different formulations to the animal. Thus, when the active ingredients are combined into a single formulation, the formulation provides protection against a broader spectrum of parasites than a formulation containing any single parasitical agent. As used herein, the identification of an active ingredient, e.g., pyrantel pamoate or ivermectin, is intended to cover pharmaceutically active forms thereof such as salts, hydrochlorides, chelates, and so forth.

Accordingly, in one preferred embodiment of the invention, the multidrug composition comprises at least two active ingredients. In a preferred embodiment of the invention, the first active ingredient in the composition comprises a macrocyclic lactone, which is preferably ivermectin, and the second active ingredient comprises a hexahydropyrazinoisoquinoline, which is preferably praziquantel.

In another preferred embodiment of the invention, the multidrug composition comprises three active ingredients that are delivered in a palatable form. The first active ingredient in the composition comprises a macrocyclic lactone such as ivermectin, selamectin, dormectin, moxidectin, eprinomectin, and abamectin, the second active ingredient comprises a tetrahydropyrimidine such as pyrantel pamoate, morantel and oxantel, and the third active ingredient in the composition comprises a hexahydropyrazinoisoquinoline, preferably, praziquantel.

In another preferred embodiment of the invention, the first active ingredient comprises ivermectin, the second active ingredient comprises pyrantel pamoate, and the third active ingredient comprises praziquantel.

A particular problem encountered in the preparation of multidrug compositions is that some active ingredients interact with one another, resulting in the production of an unstable pharmaceutical composition. In particular, ivermectin can interact with other active ingredients thereby producing a composition that can be unstable when stored at room temperature. Therefore, the composition should be prepared in a manner so as to prevent ivermectin from interacting with other active ingredients in the composition. It should be noted that while the present invention is directed to multidrug anthelmintic formulations comprising parasitical agents, this method can be used to prepare a composition containing other lipid compatible active ingredients that would typically interact with each other.

Accordingly, one embodiment of the invention is directed to a method of producing a stable multidrug composition such composition can be highly effective against a variety of helminths. Preferred embodiments of the invention involve isolating the adversely reactive ingredients in a lipid base or separating the materials with layers of lipid instead. The use of lipid materials for forming solid-like pharmaceutical compositions is discussed in U.S. Pat. Nos. 6,541,025 and 6,340,471, the contents of which are incorporated herein by reference. Neither patent describes the procedure of using a lipid layering method to separate active ingredients which will degrade or otherwise interact with one of the others. Rather, U.S. Pat. No. 6,340,471 is concerned with taste masking and controlled release during delivery, not preventing interaction of active ingredients.

One preferred method of preparing of the multidrug composition comprises:
(a) preparing a first and optionally also a second and/or third liquid lipid suspension comprising a first, second, and optionally a third active ingredient, respectively, at a temperature high enough to maintain the suspension in a pourable condition;
(b) pouring the first liquid lipid suspension into a container;
(c) permitting the first lipid suspension to cool, thereby resulting in a first solid-like layer comprising the first active ingredient;
(d) optionally pouring the second lipid suspension at a high enough temperature to be liquid-like, on the first solid layer;
(e) permitting the second lipid suspension to cool, thereby resulting in a second solid-like layer located on top of the first solidified layer;
(f) optionally pouring a third liquid-like lipid suspension at a temperature high enough to be pourable on top of the second solid layer; and
(e) permitting the third liquid lipid suspension to cool, thereby resulting in a third solidified layer located on top of the second solid layer, wherein the first active ingredient in the first layer is separated from the third active ingredient in the third layer by the second solid layer.

In one embodiment of the invention, the second layer is only a separation layer, and containing no active ingredients.

As used herein, terms such as solid, melting, liquid, and so forth are not given their strict technical definitions, as many lipid compositions may not have definite melting points and freezing points. A skilled artisan will appreciate that many lipids are referred to as solid at approximately room temperature, in that they exhibit many solid-like characteristics, yet are liquid or pourable at a higher temperature. In the present invention, it is intended that the “melted” or “liquid” lipids will be sufficiently liquid so that solids can be uniformly distributed through the lipid. Accordingly, it is expected that those of ordinary skill in the art will understand what is meant by a solid, solid-like, liquid or melted lipid suspension.

In a preferred embodiment of the invention, the first active ingredient in the multidrug composition comprises ivermectin, the second active ingredient comprises pyrantel pamoate, and the third active ingredient comprises praziquantel, wherein at least ivermectin and optionally also pyrantel pamoate and praziquantel are contained in discrete, separate layers of the composition and a layer, such as a layer containing pyrantel pamoate, prevents ivermectin from interacting with praziquantel.

The first step in the process of preparing the multidrug composition includes preparing a first lipid containing suspension comprising a first active ingredient, preferably ivermectin. A second and third lipid containing suspension comprising a second and third active ingredient, preferably pyrantel pamoate and praziquantel, respectively, is prepared using the same procedure. The active ingredient is isolated and dried prior to mixing the active ingredient with the lipid containing composition. In one preferred embodiment of the invention, the active ingredient, ivermectin, pyrantel pamoate or praziquantel, is dry-blended with other dry components of the formulation such as flavors.

In another preferred embodiment of the invention, the active ingredient, ivermectin, pyrantel pamoate or praziquantel, is encapsulated by coating the active ingredient with a polymer such as ethyl cellulose. In a preferred embodiment of the invention, the active ingredients are encapsulated by pan coating.

In a preferred embodiment of the invention, lipids are used as the carrier vehicle for the active ingredient. The lipids are melted by heating to the appropriate temperature, preferably below the decomposition temperature of the active ingredient that is to be mixed into the pourable lipid mixture. In a preferred embodiment of the invention, a surfactant is included in the composition, which can be either dry blended with the active ingredient prior to mixing with the melted lipids or added to the pourable lipid mixture.

The pouring temperature of the lipid mixture should be high enough to permit convenient pouring, but should not be so high as to reliquidify any already existing layers. Accordingly, in one embodiment of the invention, successive layers are formed with lower liquidification points, so that the existing solidified layers do not unduly soften. In another preferred embodiment of the invention, the solidified layer(s) are chilled prior to pouring successive lipid layers in order to prevent the melting or softening of a solidified layer resulting from the pouring of a successive lipid layer.

In a preferred embodiment of the invention, the melting point of the lipid used to suspend the active ingredient should not be below 90° F. and should not be above 150° F. The source of lipids can consist of a single component “hard butter”, which refers to a lipid system that has characteristics and/or a solid fat melting index similar to cocoa butter and is similar in rapid meltdown characteristics. Typical lipids include, but are not limited to, partially hydrogenated vegetable oil, soybean oil, cottonseed oil, palm oil and palm oil and palm kernel oil. The lipid system can also comprise petroleum wax, vegetable or animal stearines, or a high solids sharp melting point vegetable fat, or also combinations of hard butters and stearines. It is also possible to use mineral oil or petrolatum as the liquid hydrophobic system.

In an alternative embodiment of the invention, matrix materials other than lipids can be used as the carrier vehicle for the active ingredient. The carrier system can be prepared using polymers such as gelatin, agar, carrageenan and gellan, all of which can be dissolved in water by heating, and solidified to a gel by cooling.

In another alternative embodiment of the invention, alginate solutions can also be used, which can be solidified to a gel by the addition of calcium (Ca²⁺) ions. This gelation of the solution can be controlled by the incorporation of the appropriate amounts of phosphate and calcium ions. Alginate solutions can be utilized as the carrier vehicle without the use of heat, and therefore, alginate solutions are useful for active ingredients that are heat labile or thermally sensitive. Accordingly, in one embodiment of the invention, a pharmaceutical composition is provided that comprises a first active ingredient in a lipid layer and a second active ingredient in a lipid alternative layer.

The dried particles, which may include the active ingredient, filler and optional flavorings and additives, are added to the heated lipid base to produce a liquid lipid suspension. The flavoring additives may include, for example, cheese, peanut butter, ground animal protein, synthetic flavoring, and/or flavor enhancers such as monosodium glutamate.

In a preferred embodiment of the invention, the active ingredient is added to the melted lipid solution by simple mixing in order to uniformly distribute the active ingredient throughout the liquid lipid suspension. The dry particles should be smaller than 250 microns in order to achieve uniform suspension in the lipid mixture. The dry particles of active ingredient are added slowly in order to produce an advantageously homogeneous liquid lipid suspension, i.e., no agglomerated clumps of active ingredient should be formed in the lipid suspension. Slow addition of the active ingredients will ensure that the resulting melted lipid suspension that is pumpable and flowable.

In a preferred embodiment of the invention, the concentration of the active ingredient in the lipid suspension is below 40% so as to maintain the mechanical properties of the dosage form. High solids loading will affect the mouthfeel of the composition and hence the palatability of the composition to the animal. Therefore, maintaining the concentration of the active ingredients in the suspension below 40% will ensure that the composition is delivered in a palatable form to the animal. Preferably, this mixing step is accomplished in a heated mixing device that insures thorough mixing of all materials.

The same procedure is used to produce a second and third liquid lipid suspension comprising a second and third active ingredient, preferably pyrantel pamoate and praziquantel, respectively.

Optionally, once the dry particles of active ingredient are fully dispersed in the liquid lipid mixture, the first liquid lipid suspension is cooled to a temperature just slightly higher than the solidification point of the lipid base.

The first liquid lipid suspension containing ivermectin is then poured into a mold or container for production into a final dosage form. After it is poured into the container, the first liquid lipid layer containing ivermectin is cooled to a solid. The second liquid lipid layer containing pyrantel pamoate is then poured over at least a portion of the first lipid layer containing ivermectin and cooled to a solid. The second lipid layer should not be at such a high temperature that is melts the first layer. Lastly, the third liquid lipid layer containing praziquantel is poured over at least a portion of the second lipid layer containing pyrantel pamoate and cooled to a solid.

It should be noted that the manner in which the second liquid lipid layer is poured over the first lipid layer is important in the preparation of the multidrug composition of the present invention. The second lipid layer must be poured on top of the cooled first lipid layer in a manner so that once cooled, the second lipid layer advantageously covers the entire surface of the cooled first lipid layer in the container. The second lipid layer can contain advantageously contain a non-reactive active ingredient such as pyrantel pamoate, which does not react with or degrade either ivermectin or praziquantel. Accordingly, it is this second lipid layer that physically separates the first and third lipid layers and thereby prevents the interaction of ivermectin in the first lipid layer with praziquantel in the third lipid layer.

This approach of producing a multidrug composition may also be utilized if the dosage form requires only two actives that interact with each other. In such an event, the first and third lipid layers can each contain an active ingredient and the second lipid layer can be prepared without an active ingredient. The second lipid layer is placed between the first and third lipid layers containing the active ingredients in order to prevent the active ingredients from interacting. The use of this second lipid layer as a physical barrier between the active ingredients, preferably ivermectin and praziquantel, thereby produces a multidrug composition that is stable and highly effective against a variety of helminths.

It should be understood that this approach of preparing a multidrug composition comprising of separate multiple layers of active ingredients contained in a lipid/phospholipid vehicle can be extended to cover more than two active ingredients that interact. For example, in one embodiment of the invention, one or more active ingredients can be encapsulated prior to incorporation into one of the liquid lipid layers.

In one preferred embodiment of the invention, the composition comprises ivermectin, pyrantel pamoate and praziquantel for the treatment of helminths. Preferably, the composition comprises approximately about 65 to 275 micrograms of ivermectin, 160 to 675 milligrams of pyrantel pamoate and 50 to 250 milligrams of praziquantel.

In one preferred embodiment of the invention, a composition comprising approximately 272 micrograms ivermectin, 656 milligrams pyrantel pamoate and 228 milligrams praziquantel can be administered to a dog weighing over 60 pounds.

In another preferred embodiment of the invention, a composition comprising approximately 136 micrograms ivermectin, 344 milligrams pyrantel pamoate and 114 milligrams praziquantel can be administered to a dog weighing between 20 and 60 pounds.

In another preferred embodiment of the invention, a composition comprising approximately 68 micrograms ivermectin, 164 milligrams pyrantel pamoate and 57 milligrams praziquantel can be administered to a dog weighing less than 20 pounds.

A preferred dosage of avermectin, e.g., ivermectin, is about 4-20 μg/Kg body weight of the animal administered monthly. It should be of course understood that the actual amount of ivermectin will vary depending upon the size of the animal being treated. In a preferred embodiment of the invention, the preferred dosage of ivermectin for dogs weighing 60 to 150 lbs (27-68 kg) is approximately 272 μg, the preferred dosage of ivermectin for dogs weighing 20 to 60 lbs (9-27 kg) is approximately 136 μg, and the preferred dosage of ivermectin for dogs weighing less than 20 pounds (less than 9 kg) is approximately 68 μg.

A preferred dosage of anthelmintic pyrimidines, e.g., pyrantel pamoate, is about 5-50 mg/Kg body weight administered monthly. A preferred dosage of hexahydropyrazinoisoquinaline, e.g., praziquantel, is about 3-19 mg/Kg body weight administered monthly. It should be of course understood that the actual amount of active ingredients will vary depending upon the size of the animal being treated.

The antiparasitic agents of this invention find their primary use in the treatment and/or prevention of helminthiasis; however, they may also useful in the prevention and treatment of diseases caused by other parasites. Repeat treatments are given as required to combat re-infestations and are dependent upon the particular helminth. The techniques for administering these materials to animals are known to those skilled in the field of veterinary medicine.

The preparations are suitable for combating pathogenic endoparasites which occur in animal husbandry and animal breeding in productive, breeding, zoo, laboratory, experimental animals and pets, and have a favorable toxicity to warm-blooded animals. In this connection, they are active against all or individual stages of development of the pests and against resistant and normally sensitive species. By combating pathogenic endoparasites, it is intended that disease, cases of death and reduction in production (for example in the production of meat, milk, wool, hides, eggs, etc.) are reduced so that more economic and simpler animal husbandry is possible by means of the use of the pharmaceutical formulation.

Productive and breeding animals include mammals, such as, for example, cattle, horses, sheep, pigs, goats, camels, water buffalo, donkeys, rabbits, fallow deer and reindeer, pelt animals, such as, for example, mink, chinchilla and raccoons, birds, such as, for example, chickens, geese, turkeys and ducks, fresh and salt-water fish, such as, for example, trout, carp and eels, and reptiles.

Laboratory and experimental animals include mice, rats, guinea pigs, golden hamsters, dogs and cats.

Pets include dogs and cats.

The formulation according to the invention is particularly preferably administered to dogs and cats, but is suitable for other mammals.

Administration can take place both prophylactically and therapeutically.

The formulations of the invention are intended to be orally administered.

Auxiliaries can include preservatives, antioxidants and colorants. Additional suitable auxiliaries can include lubricants, such as, for example, magnesium stearate, stearic acid, talcum and bentonites, disintegration-promoting substances, such as starch or transversely crosslinked polyvinyl pyrrolidone, binders, such as, for example, starch, gelatin or linear polyvinyl pyrrolidone, and dry binders, such as microcrystalline cellulose.

The formulation can also be in the form of a chewable, such as a beef-chewable containing ground or minced beef or other meat, in addition to other excipients listed above.

The materials in the final formulation, such as the excipients, auxiliaries, synergists and other materials, which aid in delivery, shelf-life, desired physical structure and so forth will be referred to herein generally as carrier material. As stated herein, carrier material could be pharmaceutically active under certain circumstances.

The following examples are given for purposes of illustration only and are not intended to be construed in a limiting manner.

### EXAMPLE 1

### Preparation of Dry Blend of Ivermectin, Praziquantel and Pyrantel Pamoate

Dried granules of ivermectin, praziquantel and pyrantel pamoate were sieved to a particle size of less than 250 microns.

### EXAMPLE 2

### Forming the Lipid Suspension

The first lipid, a vegetable stearine (Duratex®) was placed into a kettle and heated in the range of about 140 to 150° F. and melted. A second lipid, vegetable hard butter, (Kalomel®) was added to the first lipid and allowed to melt. A surfactant, lecithin, was added to the melted lipids with mixing, and the mixture is allowed to cool to about 135° F.

The ivermectin dry particles according to the procedure of Example 1 were slowly added incrementally to the lipid/surfactant mixture with mixing over a period of about 1 hour, to provide a smooth suspension with no lumps or agglomerations. The final concentration of ivermectin in the lipid suspension is less than 40%.

The above approach is repeated to prepare a second and third liquid lipid layer containing the second and third active ingredients, pyrantel pamoate and praziquantel, respectively.

### EXAMPLE 3

### Pouring the Lipid Layers and Forming the Multidrug Composition

The first liquid lipid suspension containing ivermectin was poured into a mold and allowed to cool to a solid. The second liquid lipid layer suspension containing pyrantel pamoate was carefully poured on top of the cooled lipid layer containing ivermectin. Care was taken to ensure that the entire surface of the first lipid layer was covered by the second lipid layer. After the second lipid layer containing pyrantel pamoate was cooled to a solid, the third liquid lipid layer was poured on top of the second lipid layer and cooled to a solid.

### EXAMPLE 4

### An Exemplary Formulation with Different Flavoring Agents

Table 1 shows an example of active ingredients in combination with different flavoring agents and additives for formulations in accordance with preferred embodiments of the invention. The palatability of unflavored formulation, defined as the percentage of dogs that ingested all the tablets, was found to be 55%. The addition of cheese flavor raised palatability to 75%. The addition of peanut butter only raised palatability to only 65%. However, adding DPPE, described below, raised palatability from 65% to 80%.

TABLE 1

| | | | Peanut Butter + | | Peanut | | | |
|---|---|---|---|---|---|---|---|---|
| | Cheese | | DDPE | | Buffer Only | | No Flavors | |
| | Weight | | Weight | | Weight | | Weight | |
| Ingredients | (mg) | % | (mg) | % | (mg) | % | (mg) | % |
| Ivermectin | 0.136 | 0.0068% | 0.136 | 0.0063% | 0.136 | 0.0069% | 0.136 | 0.0078% |
| Pyrantel | 344.00 | 17.200% | 344.00 | 15.897% | 344.00 | 17.515% | 344.00 | 19.770% |
| pamoate | | | | | | | | |
| Coated | 240.00 | 12.000% | 240.00 | 11.091% | 240.00 | 12.220% | 240.00 | 13.793% |
| praziguantel | | | | | | | | |
| Klx | 368.00 | 18.400% | 368.00 | 17.006% | 368.00 | 18.737% | 368.00 | 21.150% |
| Sterene 27 | 368.00 | 18.400% | 368.00 | 17.006% | 368.00 | 18.737% | 368.00 | 21.150% |
| Lecithin | 20.00 | 1.000% | 20.00 | 0.924% | 20.00 | 1.018% | 20.00 | 1.149% |
| Acdisol | 199.93 | 9.996% | 199.93 | 9.239% | 199.93 | 10.180% | 199.93 | 11.490% |
| Explotab | 199.93 | 9.996% | 199.93 | 9.239% | 199.93 | 10.180% | 199.93 | 11.490% |
| Cheese flavor | 40.00 | 2.000% | 0.00 | 0.000% | 0.00 | 0.000% | 0.00 | 0.000% |
| DDPE | 200.00 | 10.000% | 200.00 | 9.242% | 0.00 | 0.000% | 0.00 | 0.000% |
| Peanut butter | 0.00 | 0.000% | 210.00 | 9.704% | 210.00 | 10.693% | 0.00 | 0.000% |
| flavor | | | | | | | | |
| Salt | 20.00 | 1.000% | 10.00 | 0.462% | 10.00 | 0.509% | 0.00 | 0.000% |
| Sucralose | 0.00 | 0.000% | 4.00 | 0.185% | 4.00 | 0.204% | 0.00 | 0.000% |
| Total | 1999.99 | 100.00% | 2163.99 | 100.00% | 1963.99 | 100.00% | 1739.99 | 100.00% |

| | | | | |
|---|---|---|---|---|
| Palatability % | 75 | 80 | 65 | 5 |

As shown in Table 1, the formulation may contain various flavoring agents, additive, and excipents. The DDPE is a flavor enhancer with the registered trade name “Optimizor” from Applied Food Biotechnology, Inc., O'Fallon, Mo. It is a blend of vegetable derived ingredients that enhance palatability of formulations for dogs. The Table 1 formulations contain cheese or peanut butter as flavorings. Other flavorings such as beef, poultry, pork, lamb, both synthetic and naturally-derived, or ground beef, chicken, turkey, pork and lamb may be included in the formulation. Flavor enhancers such as monosodium glutamate or other ground crunchy material such as kibble or ground biscuit may also be added. The range of flavoring agents or flavor enhancers preferably ranges from 10% to 19%.

It will thus be seen that the objects set forth above, among those made apparent from the preceding description, are efficiently attained and, since certain changes may be made in carrying out the above method and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall therebetween. Particularly it is to be understood that in said claims, ingredients or compounds recited in the singular are intended to include compatible mixtures of such ingredients wherever the sense permits.

What is claimed is:

1. A method for forming a multilayered anthelmintic formulation, comprising the steps of:
(a) preparing a first, second and third liquid-like lipid containing suspension, each comprising a first, second, and third active ingredient, respectively;
(b) transferring the first liquid-like lipid suspension into a container;
(c) permitting the first liquid-like lipid suspension to cool, thereby resulting in a first solid-like layer comprising the first active ingredient;
(d) disposing the second lipid-like suspension over the first solid-like layer to cover at least a portion of the surface of the first solid-like layer with the second lipid-like suspension;
(e) permitting the second liquid lipid-like suspension to cool, thereby resulting in a second solid-like layer located over the first solid-like layer;
(f) disposing the third liquid lipid-like suspension over at least a portion of the second solid-like layer; and
(e) permitting the third liquid-like lipid suspension to cool, thereby resulting in a third solid-like layer located over the second solid-like layer, wherein the first active ingredient in the first solid-like layer is separated from the third active ingredient in the third solid-like layer by the second solid-like layer.

2. The method of claim 1, wherein the preparation of each of the first, second and third liquid lipid suspensions comprises (i) melting at least one lipid, (ii) drying the active ingredient, and (iii) mixing the dried active ingredient into said melted lipid.

3. The method of claim 2, wherein the drying step comprises dry-blending.

4. The method of claim 1, wherein the first active ingredient comprises ivermectin.

5. The method of claim 4, wherein the second active ingredient comprises pyrantel pamoate.

6. The method of claim 5, wherein the third active ingredient comprises praziquantel.

7. The anthelmintic formulation which is formed by the method of claim 1.

8. The anthelmintic formulation which is formed by the method of claim 4.

9. The anthelmintic formulation which is formed by the method of claim 6.

10. A pharmaceutical formulation comprising a first lipid containing layer comprising a first active ingredient; a second lipid containing layer over the first lipid containing layer; and a third lipid containing layer separated from the first layer by the second layer, the third layer comprising a second active ingredient that reacts or is degraded by the first active ingredient.

11. The formulation of claim 10, wherein the first active ingredient comprises ivermectin and the second active ingredient comprises praziquantel.

12. The formulation of claim 11, wherein the second lipid containing layer comprises pyrantel pamoate.

13. The formulation of claim 11, wherein the second lipid layer prevents ivermectin from interacting with praziquantel.

14. The formulation of claim 13, wherein the formulation comprises about 65 to 275 micrograms ivermectin.

15. The formulation of claim 14, wherein the formulation comprises about 160 to 275 milligrams pyrantel pamoate.

16. The formulation of claim 15, wherein the formulation comprises about 50 to 250 milligrams praziquantel.

17. The formulation of claim 16, wherein the formulation comprises about 272 micrograms ivermectin, 656 milligrams pyrantel pamoate and 228 milligrams praziquantel.

18. The formulation of claim 16, wherein the formulation comprises about 136 micrograms ivermectin, 344 milligrams pyrantel pamoate and 240 milligrams praziquantel.

19. The formulation of claim 16, wherein the formulation comprises about 68 micrograms ivermectin, 164 milligrams pyrantel pamoate and 57 milligrams praziquantel.

20. A method for treating or preventing helminthiasis in mammals, said method comprising administering an anthelmintically effective amount of the formulation of claim 10 to the mammal.

21. The method of claim 20, wherein the mammal is a cat or a dog.

22. A method for treating or preventing helminthiasis in mammals, said method comprising administering an anthelmintically effective amount of the formulation of claim 12 to the mammal.

23. The method of claim 22, wherein the mammal is a cat or a dog.

24. A method for treating or preventing helminthiasis in mammals, said method comprising administering an anthelmintically effective amount of the formulation of claim 16 to the mammal.

25. The method of claim 24, wherein the mammal is a cat or a dog.

26. A method for treating or preventing helminthiasis in mammals, said method comprising administering an anthelmintically effective amount of the formulation of claim 17 to the mammal.

27. The method of claim 26, wherein the mammal is a cat or a dog.

28. A method for treating or preventing helminthiasis in mammals, said method comprising administering an anthelmintically effective amount of the formulation of claim 18 to the mammal.

29. The method of claim 28, wherein the mammal is a cat or a dog.

30. A method of forming a pharmaceutical formulation containing at least two active ingredients, comprising:
(a) combining a first active ingredient with a lipid containing composition and forming a pourable first active composition;
(b) allowing the first active composition to cool to form a first active layer;
(c) forming a hardened second lipid containing layer adjacent the first active layer;
(d) combining a second active ingredient with a second lipid containing composition and forming a pourable second active composition; and
(e) forming a cooled, hardened layer of the second active composition over at least a portion of the second lipid containing layer.

31. The method of claim 30, wherein the first active ingredient is ivermectin and the second active ingredient is praziquantel.

32. The method of claim 31, wherein the hardened second lipid containing layer comprises pyrantel pamoate.

33. The pharmaceutical formulation formed by the method of claim 30.

34. The pharmaceutical formulation formed by the method of claim 31.

35. The pharmaceutical formulation formed by the method of claim 32.
